(19) United States
(12) Patent Application Publication
(10) Pub. No.: US 2003/0065381 A1
(43) Pub. Date: Apr. 3, 2003

(54) Longitudinal focussed force stent

(76) Inventors: Ronald J. Solar, San Diego, CA (US); Glen L. Lieber, Poway, CA (US)

Correspondence Address: WILLIAM H. DIPPERT, REED SMITH LLP, 599 LEXINGTON AVENUE, 29TH FLOOR, NEW YORK, NY, 10022-7650, US

(21) Appl. No.: 09/965,770

(22) Filed: Sep. 28, 2001

(51) Int. Cl. A61F 2/06

(52) U.S. Cl. 623/1.15

(57) ABSTRACT

A device of the present invention includes a generally tubular stent body with one or more external longitudinal projections. The stent is used for insertion into a vessel. These projections may extend from the distal end of the stent to the proximal end of the stent, or they may terminate at a location proximal to the distal end of the stent and/or distal to the proximal end of the stent. The projections act as rails to reduce a contact area between the stent and a vessel wall as well as act to focus and concentrate the radial forces. Preferably the distal end of each projection is tapered to facilitate crossing a tight undilated stenotic segment. When the stent is inserted into the vessel, it is expanded by balloon inflation, shape memory, self-expansion and, other means. The projections may be formed in the stent, added as separate elements and attached by suitable methods, or formed by crimping the stent with a suitable tool.

### FIELD OF THE INVENTION

The invention relates generally to an improved stent that is particularly suitable for use with tight strictures. More particularly, the invention relates to the stent and its use in a stenting procedure where predilation of a stenosis is not performed.

### BACKGROUND OF THE INVENTION

The use of stents to support anatomical cavities, passageways, or blood vessel segments is well known. The most common application of stents is in stenotic or narrowed blood vessels that have been treated by balloon angioplasty. In balloon angioplasty a balloon dilatation catheter is used to dilate a stenotic portion of a blood vessel. Such dilation stretches the vessel wall in such a manner as to cause an injury to the vessel wall that allows the dilated portion of the blood vessel to remain enlarged.

Healing of the injured blood vessel results in long-term patency of the treated vessel segment. However, in a large percentage of patients, the dilated blood vessel segment re-narrows, which is known as restenosis. To reduce the occurrence of restenosis, stents are frequently placed into a dilated blood vessel segment immediately following balloon angioplasty.

Recently, for a variety of procedural and clinical reasons, there has been an increased interest in deploying a stent without prior dilation of the stenotic area, or lesion, of a blood vessel. In spite of the perceived benefits of this technique, there are a number of problems to overcome in placing and deploying a stent without predilation. For example, most stents have slots or other cutout configurations in their respective walls. The edges of these slots may drag or catch against the narrowed blood vessel segment and prevent the passage of the stent, may cause stent displacement from the delivery catheter, may break off plaque that can subsequently embolize distally in the blood vessel, or may damage the cells that line the vessel wall. In addition, without prior dilation, high balloon pressures may be required to deploy, i.e., expand, the stent since the balloon not only has to overcome the mechanical forces that are required to expand the stent, but it must also be able to dilate the stenosis. In addition to the possibility of balloon failure, laboratory and clinical studies have shown that there is an increased risk of blood vessel injury when very high balloon pressures are used to dilate stenoses.

There is thus a need to develop a device that overcomes the above problems and facilitates stent placement and deployment in stenoses that have not been predilated.

### OBJECTS OF THE INVENTION

It is an object of this invention to provide an improved stent for insertion into a vessel.

It is also an object of this invention to provide a stent useful for insertion into a vessel without prior dilation of said vessel.

It is a further object of this invention to provide an improved stent for insertion into a vessel which enables the application of focused forces on the vessel walls.

It is yet a further object of this invention to provide an improved stent for insertion into a vessel which allows for the use of lower pressure dilation within the vessel.

These and other objects of the invention will become more apparent from the following more detailed discussion provided below.

### SUMMARY OF THE INVENTION

A device of the present invention comprises a generally tubular stent body with one or more external longitudinal projections, for insertion into a corporeal lumen such as a blood vessel. These projections may extend from the distal end of the stent to the proximal end of the stent, or they may terminate at a location proximal to the distal end of the stent and/or distal to the proximal end of the stent. The projections act as rails to reduce the contact area between the stent and a vessel wall, as well as to focus the radial forces. The distal end of each projection is tapered to facilitate crossing a tight, undilated stenotic segment. When the stent is inserted into the vessel, it is expanded by applied expansion, such as balloon inflation, self-expansion, or other means known in the art.

As the stent of the invention expands against a stenosis, the projections on the stent act as stress concentrators due to their smaller contact area. Thus, for a given balloon inflation pressure or stent expansion force, the stresses at the portion of the stenosis in contact with the projections are greatly magnified, thus allowing the stenosis to expand at a lower balloon pressure than if the projections were not present. This lower pressure dilation may result in diminished vessel injury and diminished procedural complications.

The projections may be formed in the stent, formed as separate elements and attached later by suitable methods, or formed by crimping the stent with a suitable tool.

### BRIEF DESCRIPTION OF THE DRAWINGS

The advantages of the invention will be apparent upon consideration of the following detailed description, taken in conjunction with the accompanying drawings:

FIGS. 1, 2, 3, and 4 are each a schematic representation of an embodiment of a stent according to the invention;

FIG. 5 is a schematic representation of a cross-sectional view of a stent according to FIG. 1 along line 5-5;

FIG. 6 is a perspective schematic representation of one end of a stent according to the invention having three longitudinal projections, each having a tapered end configuration;

FIG. 7 is a schematic representation of a cross-sectional view of an unexpanded stent according to the invention inside a blood vessel;

FIG. 8 is a schematic representation of a cross-sectional view of an expanded stent according to the invention inside a stricture or stenosis in a blood vessel.

FIG. 9 is a schematic representation of a cross-sectional view of an unexpanded prior art stent inside a blood vessel.

FIG. 10 is a schematic representation of a cross-sectional view of an expanded prior art stent according to the invention inside a stricture or stenosis in a blood vessel.

FIG. 11 is a schematic representation of a cross-sectional view of a stent according to the invention before a crimping or other forming operation to create the longitudinal projections; and

FIG. 12 is a schematic representation of a cross-section view of a stent according to the invention after a crimping or other forming operation to create the longitudinal projections.

### DETAILED DESCRIPTION OF THE INVENTION

A stent of the present invention includes a generally tubular body having one or more external longitudinal projections or members. These projections optionally may extend from the distal end of the stent to the proximal end of the stent, or they may terminate at a location proximal to the distal end of the stent and/or distal to the proximal end of the stent. Generally speaking the structure of the stent according to the invention may take any of a number of configurations as are generally known in the art. While a preferred embodiment incorporates stent designs that use a balloon to expand the stent, the present invention includes stent designs that employ means of expansion other than balloon expansion, for example, self-expanding, resilient materials, shape memory, mechanical means, and other means.

The stent body shown in the drawings here can correspond to any stent construction that is otherwise suitable for the intended purpose. A primary criterion is that the stent body provides a stable foundation for the projections. For example, a stent body could comprise a slotted tube, coil, lattice-work, zig-zag, or serpentine construction. Examples of stent designs and structures that could be used in the present invention include, but are not limited to those taught in U.S. Pat. Nos. 5,133,732, 5,158,548, 5,382,261, 5,236,446, 5,656,036, 5,683,453, and 5,413,557, the teachings of each of which are incorporated herein by reference. Commercial embodiments of devices disclosed in these patents are sold under trade names MULTILINK®, PALMAZ-SCHATZ®, GIANTURCO-ROUBIN, DUET®, CROWN®, CROSSFLEX®, BX VELOCITY®, WIKTOR®, MICRO®, S540™, and others.

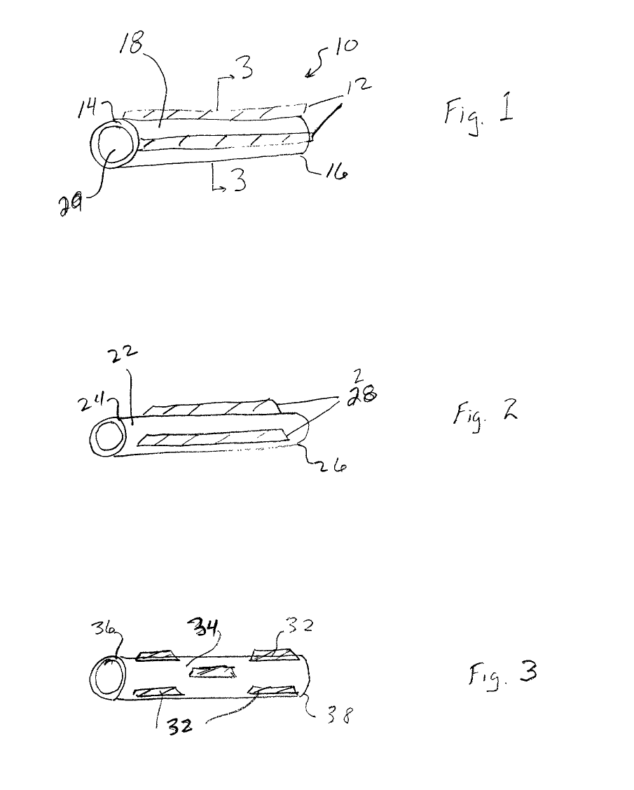

The invention can perhaps be better appreciated from the drawings. In FIG. 1 a stent 10 has three longitudinal projections or members 12 extending from the proximal end 14 to the distal end 16 of a stent body 18 having a lumen 20. The projections 12 extend radially out from the wall surface of stent body 18, i.e., out of the plane of the stent wall surface, and extend in the same direction as the longitudinal axis of stent 10. Alternatively, as shown in FIG. 2, projections or members 28 may be placed on a stent body 22 in such manner as not to fully extend to the proximal end 24 and/or distal end 26 of stent body 22. Also, as shown in FIG. 3, smaller projections 32 can be arranged in rows or other configurations on stent body 34 in the space between proximal end 36 and distal end 38. Projections 32 are preferably all positioned coextensively with the longitudinal axis of stent body 34. It is within the scope of the invention that projections 32 could be sufficiently small so as to constitute “points” or “dots”, so long as there are a sufficient number and size of the smaller projections or points to function as a rail and to concentrate radial forces.

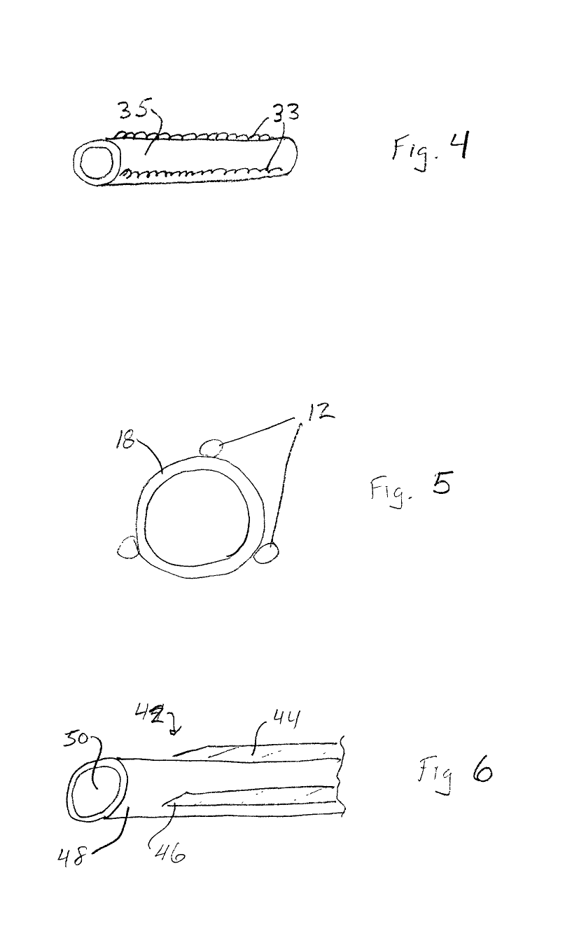

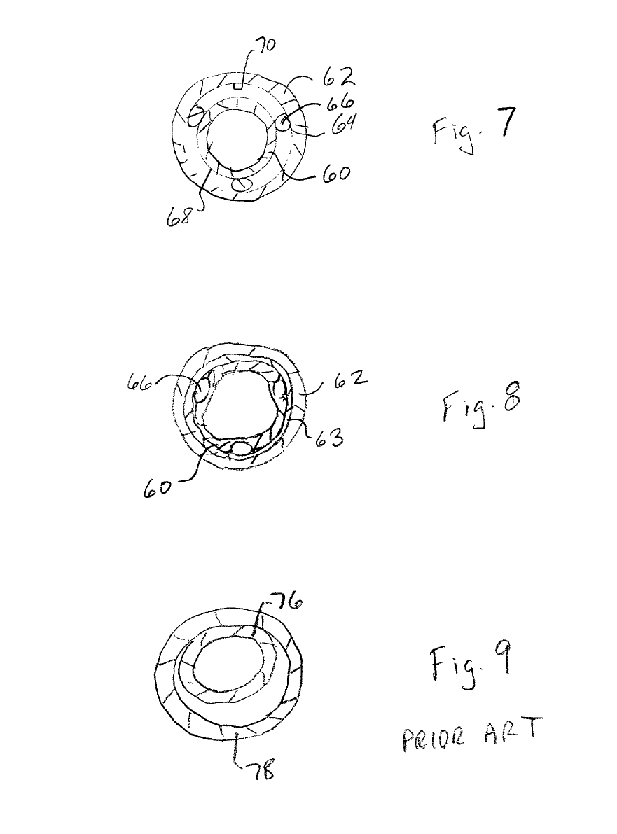

The longitudinally extending projections may have variously shaped cross-sectional geometry. It is within the scope of the invention that a projection cross-section may be circular, semi-circular, rectangular, triangular, trapezoidal, or square, or any other suitable shape, as would be appreciated by those skilled in the art. If the cross-section has a sharp surface or edge extending outward, it is preferable that a sheath be used during advancement of the stent to the desired location. The sheath may be of any suitable design as known in the art. There should be at least one, preferably from 3 to 5, projections with similar cross-sections, preferably equidistantly-spaced around the circumference of the outer surface of the stent body. In a preferred embodiment of the invention, there are three, equidistantly-spaced projections. In addition, as illustrated in FIG. 3, there may be multiple projections along the length of the stent. Such an arrangement may provide for improved flexibility in delivering longer stents. Alternatively, the projections themselves may be flexible. For example, as illustrated in FIG. 4, each projection may take the form of a flexible, helically wound coil 33 positioned on stent body 35.

FIG. 5 represents a cross-sectional view of FIG. 1, where it can be seen that there are three equidistantly-spaced projections 12, each of which has a circular cross-section.

The projections provided for may optionally be formed integrally or may be attached by suitable means, for example, by use of solder, braze, weld, adhesives, and other means. Preferably the projections are formed integrally with the stent. It is preferred that the opposite ends, or at least the distal end, of the projections are formed tapered. Such tapered construction is illustrated in FIG. 6, where stent 42 has projections 44 with tapered ends 46, arranged on stent body 48 having a lumen 50.

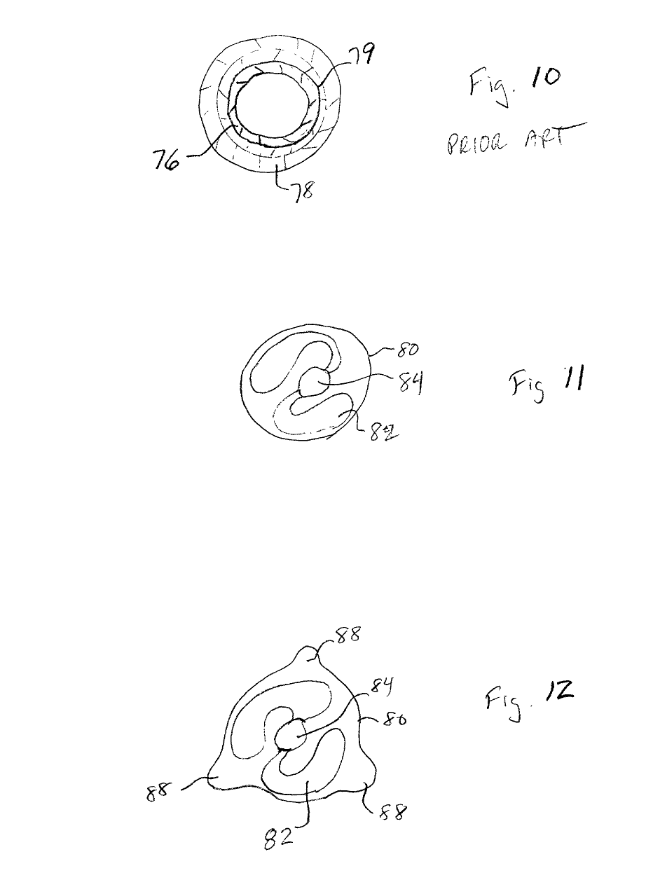

The stent according to the present invention includes at least one longitudinal projection external to the outer surface of the stent. In a preferred embodiment of the invention, the stent has at least three projections spaced equidistantly around the circumference of the (unexpanded) stent. As shown in FIG. 7 where an unexpanded stent 60 is positioned within a blood vessel 62, the outer surface 64 of each projection 66 is significantly less in contact area than the outer surface 68 of stent 60, thus greatly reducing the contact area between stent 60 and the inside wall 70 of blood vessel 62. FIG. 8 illustrates the stent 60 expanded within a stenosis 63 of blood vessel 62. For comparison purposes, placement or insertion of a prior art stent is illustrated in FIGS. 9 and 10, where a prior art stent 76 is positioned within a blood vessel 78 during insertion (FIG. 9) and expanded within stenosis 79 (FIG. 10).

The distal or leading ends (FIGS. 2, 6) of the projections provided are preferably tapered to facilitate crossing tight, undilated stenotic segments. Once in place, the stent is expanded by means known in the art, for example, by balloon inflation, shape memory, self-expansion, and equivalent means. Due to the smaller contact area of the projections, as the stent expands against the stenosis the projections act as stress concentrators, such that for a given balloon inflation pressure or stent expansion force the stresses at the portion of the stenosis in contact with the projections are greatly magnified. Thus this allows the stenosis to expand at a lower pressure than if the projections were not present. As described in U.S. Pat. No. 5,413,557, incorporated herein by reference in its entirety, this lower pressure dilation may result in reduced vessel injury and diminished procedural complications.

As noted above, the longitudinal projections or members may be formed in the stent by various methods known in the art, for example by use of EDM, laser cutting, photochemical etching, wrapping, and other means. Alternatively, the projections may be added as separate elements and attached by suitable methods, such as welding, brazing, soldering, adhesive bonding, and other means. In another embodiment of the invention, projections may be formed by crimping with a suitable tool. As shown in FIGS. 11 and 12, projections may be formed in this manner at the time a stent 80 is mounted onto or surrounds a balloon 82 of a delivery catheter 84, or pre-formed prior to mounting of stent 80 onto delivery catheter 84. As shown in FIG. 12, the swaging, or forming operation adds the form of projections 88 to stent 80.

The invention may be applied to covered stents and stent grafts. In these applications, the projections may be exterior to the stent cover, or alternatively interior to the stent cover material.

While the invention has been particularly shown and described with respect to illustrative and preferred embodiments thereof, it will be understood by those skilled in the art that the foregoing and other changes in form and details may be made therein without departing from the spirit and scope of the invention that should be limited only by the scope of the appended claims.

I claim:

1. A stent for insertion into a corporeal vessel, comprising:
a stent body having proximal and distal ends and an outer surface, and
at least one longitudinal projection external to said stent outer surface,
wherein each longitudinal projection acts as a rail to reduce the contact area between the stent and the vessel wall during insertion of the stent.

2. The stent of claim 1 which has a generally circular cross-section.

3. The stent of claim 1, wherein at least one longitudinal projection extends from a point at or adjacent to the distal end of the stent to a point at or adjacent to the proximal end of the stent.

4. The device of claim 1, wherein the stent includes at least three longitudinal projections.

5. The device of claim 4, wherein said at least three projections are equidistantly spaced around the circumference of the stent.

6. The device of claim 1, wherein the distal end of each longitudinal projection is tapered.

7. The device of claim 1, wherein after the stent is inserted into the vessel, the stent is expanded by balloon inflation.

8. The device of claim 1, wherein after the stent is inserted into the vessel, the stent is expanded by shape memory.

9. The device of claim 1, wherein after the stent is inserted into the vessel, the stent is expanded by self-expansion.

10. The device of claim 1, wherein at least one longitudinal projection acts as a stress concentrator, such that for a given stent expansion force the stresses at a portion of a stenosis in contact with the longitudinal projection is greatly magnified, allowing the stenosis to expand at lower pressures than if the projection were not present.

11. The stent of claim 1, wherein at least one longitudinal projection has a circular, trapezoidal, or triangular cross-section.

12. The stent of claim 1, wherein at least one longitudinal projection is formed integral with the stent wall surface.

13. The stent of claim 1, wherein at least one longitudinal projection is attached to the stent wall surface.

14. The stent of claim 1, wherein at least one longitudinal projection is flexible.

15. The stent of claim 1, wherein at least one longitudinal projection has a helical configuration.

16. A stent for insertion into a corporeal vessel, comprising:
a stent body having proximal and distal ends on an outer surface, and
at least three projections external to said stent outer surface,
wherein each projection acts as a rail to reduce the contact area between the stent and the vessel wall.

17. The stent of claim 16 which has a generally circular cross-section.

18. The stent of claim 16, wherein at least one projection is longitudinal.

19. The stent of claim 18, wherein at least one longitudinal projection extends from a point at or adjacent to the distal end of the stent to a point at or adjacent to the proximal end of the stent.

20. The stent of claim 18, wherein the stent includes at least three longitudinal projections.

21. The stent of claim 16, wherein said at least three projections are equidistantly spaced around the circumference of the stent.

22. The stent of claim 18, wherein the distal end of each longitudinal projection is tapered.

23. The stent of claim 16, wherein after the stent is inserted into the vessel, the stent is expanded by balloon inflation.

24. The stent of claim 16, wherein after the stent is inserted into the vessel, the stent is expanded by shape memory.

25. The stent of claim 16, wherein after the stent is inserted into the vessel, the stent is expanded by self-expansion.

26. The stent of claim 18, wherein each longitudinal projection acts as a stress concentrator, such that for a given stent expansion force the stresses at a portion of a stenosis in contact with the longitudinal projection is greatly magnified, allowing the stenosis to expand at lower pressures than if the projection were not present.

27. The stent of claim 18, wherein each longitudinal projection has a circular, trapezoidal, or triangular cross-section.

28. The stent of claim 16, wherein at least one projection is formed integral with the stent wall surface.

29. The stent of claim 16, wherein at least one projection is attached to the stent wall surface.

30. The stent of claim 1, wherein at least one projection is flexible.

31. The stent of claim 1, wherein at least one projection has a helical configuration.

32. A method of magnifying stresses at a portion of a stenosis in contact with a stent, the stent including a distal end and a proximal end and having a circular cross-section, the method comprising the steps of limiting the initial contact area between a vessel wall and the stent to at least one projection, said projection being external to the surface of the stent and acting as a stress concentrator such that for a given stent expansion force, the stresses at a portion of a stenosis in contact with said projection are greatly magnified, allowing the stenosis to expand at lower pressures than if said at least one projection were not present.

33. The method of claim 32, wherein at least one projection extends from the distal to the proximal end of said stent.

34. The method of claim 32, wherein there are at least two projections equidistantly spaced around the circumference of the stent.

35. The method of claim 32, wherein each projection is tapered at the distal end to facilitate crossing an undilated stenotic segment.

36. The method of claim 32, wherein the stent comprises at least one longitudinal projection.

37. The method of claim 36, wherein the stent comprises three longitudinal projections.
